(19) United States
(12) Patent Application Publication
(10) Pub. No.: US 2025/0256104 A1
(43) Pub. Date: Aug. 14, 2025

(54) SYSTEMS AND METHODS FOR CLOSED LOOP NEUROMODULATION

(71) Applicant: NERVONIK INC., Los Angeles, CA (US)

(72) Inventor: Barry Keenan, Las Vegas, NV (US)

(21) Appl. No.: 18/858,490

(22) PCT Filed: Apr. 25, 2023
(86) PCT No.: PCT/US2023/019826

§ 371 Date: Oct. 21, 2024

Related U.S. Application Data
(60) Provisional application No. 63/335,160, filed on Apr. 26, 2022.

(51) Int. Cl. A61N 1/36; A61N 1/372

(52) U.S. Cl. CPC A61N 1/36139; A61N 1/37235

(57) ABSTRACT

A neuromodulation system includes an implantable neuromodulation device and an external unit. The neuromodulation device includes an electrode platform, a sensor/recorder coupled to the electrode platform, and a transceiver coupled to the sensor/recorder. The sensor/recorder senses electrical activity of tissue resulting from a delivery of a stimulation therapy to the tissue. The stimulation therapy is defined by a plurality of stimulation parameters, and the sensed electrical activity includes a stimulation response comprising an evoked stimulation response, a stimulation artifact, and an evoked compound action potential (ECAP) response. The transceiver transmits a sensed signal corresponding to the electrical activity. The externa unit includes a transceiver that receives the sensed signal from the neuromodulation device, and a processor that determines a measure based on the ECAP response and the evoked stimulation response; and determines whether to adjust one or more of the stimulation parameters of the stimulation therapy based on the measure.

### TECHNICAL FIELD

The present disclosure relates generally to neuromodulation systems and methods, and more particularly, to systems and methods for closed loop neuromodulation.

### BACKGROUND

Neurostimulation therapies are often limited due to a loss of therapeutic effect over time. Habituation and desensitization with increasing tolerance to the neurostimulation therapy is a considerable challenge in the treatment of disorders such as chronic pain and urinary dysfunction. This effect is exacerbated with the use of static stimulation waveforms.

The current state of the art in peripheral nerve stimulation (PNS) to treat a range of chronic disorders, such as neuropathic pain and urinary disfunction, adopt open loop modalities, wherein the pulse generator output is a fixed waveform. While this approach is sufficient to provide therapeutic benefits, it is far from optimal, with adjustments to waveform amplitude performed by the patient. Variations in the electrical field at the nerve interface where the charge is delivered to a peripheral nerve fiber are common. Variations can be a result of changes in interface impedance, electrode movement and other interfering factors. Electrode movement can result from cardiac and respiratory motion or ambulation. This can impact the ability of the device to elicit an action potential (AP) response or provide the desired stimulation response consistently.

### SUMMARY

The disclosure relates to a neuromodulation system that includes an implantable neuromodulation device and an external unit. The implantable neuromodulation device includes an electrode platform, a sensor/recorder coupled to the electrode platform, and a transceiver coupled to the sensor/recorder. The sensor/recorder is configured to sense electrical activity of tissue resulting from a delivery of a stimulation therapy to the tissue, wherein the stimulation therapy is defined by a plurality of stimulation parameters, and the sensed electrical activity includes a stimulation response comprising an evoked stimulation response, a stimulation artifact, and an evoked compound action potential (ECAP) response. The transceiver is configured to transmit a sensed signal corresponding to the electrical activity. The externa unit includes a transceiver configured to receive the sensed signal from the implantable neuromodulation device, and a processor configured to determine a measure based on the ECAP response and the evoked stimulation response; and to determine whether to adjust one or more of the plurality of stimulation parameters of the stimulation therapy based on the measure.

The disclosure also relates to a method of adjusting stimulation therapy delivered to tissue of a patient by an implanted neuromodulation device. The method includes a) sensing electrical activity of the tissue resulting from a delivery of a stimulation therapy to the tissue through an implanted neurostimulation device, wherein the stimulation therapy is defined by a plurality of stimulation parameters, and the sensed electrical activity includes a stimulation response comprising an evoked stimulation response, a stimulation artifact and an evoked compound action potential (ECAP) response. The method also includes b) determining a measure based on the ECAP response and the evoked stimulation response; and c) determining whether to adjust one or more of the plurality of stimulation parameters of the stimulation therapy based on the measure.

The disclosure also relates to an implantable neuromodulation device comprising an electrode platform, an implantable pulse generator coupled to the electrode platform, a transceiver, and a sensor/recorder coupled to the electrode platform and the transceiver. The implantable pulse generator is configured to deliver a stimulation therapy to tissue through the electrode platform, wherein the stimulation therapy is defined by a plurality of stimulation parameters. The sensor/recorder is configured to: compare the delivered stimulation therapy to an activation threshold that elicits an ECAP response for the patient; and in response to the stimulation therapy being above the activation threshold: sense electrical activity of the tissue resulting from the delivery of a stimulation therapy to the tissue wherein the sensed electrical activity includes a stimulation response comprising an evoked stimulation response, a stimulation artifact and an evoked compound action potential (ECAP) response; and transmit a sensed signal corresponding to the sensed electrical activity.

The disclosure also relates to an implantable neuromodulation device comprising an electrode platform, an implantable pulse generator, and a sensor/recorder coupled to the electrode platform. The implantable pulse generator is configured to deliver a stimulation therapy to tissue through the electrode platform, wherein the stimulation therapy is defined by a plurality of stimulation parameters. The sensor/recorder is configured to: sense electrical activity of the tissue resulting from the delivery of a stimulation therapy to the tissue wherein the sensed electrical activity includes a stimulation response comprising an evoked stimulation response, a stimulation artifact and an evoked compound action potential (ECAP) response; determine a measure based on the ECAP response and the evoked stimulation response; and determine whether to adjust one or more of the plurality of stimulation parameters of the stimulation therapy based on the measure.

The disclosure also relates to an external unit comprising a transceiver and a processor coupled to the transceiver. The transceiver is configured to receive a signal corresponding to sensed electrical activity of tissue resulting from a delivery of a stimulation therapy to the tissue by an implanted neuromodulation device. The stimulation therapy is defined by a plurality of stimulation parameters, and the sensed electrical activity includes a stimulation response comprising an evoked stimulation response, a stimulation artifact and an evoked compound action potential (ECAP) response. The processor is configured to: determine a measure based on the ECAP response and the evoked stimulation response, determine whether to adjust one or more of the plurality of stimulation parameters of the stimulation therapy based on the measure, and in response to determining to adjust one or more of the plurality of stimulation parameters, transmit stimulation information to the implanted neuromodulation device to adjust the one or more of the plurality of stimulation parameters.

It is understood that other aspects of apparatuses and methods will become readily apparent to those skilled in the art from the following detailed description, wherein various aspects of apparatuses and methods are shown and described by way of illustration. As will be realized, these aspects may be implemented in other and different forms and its several details are capable of modification in various other respects. Accordingly, the drawings and detailed description are to be regarded as illustrative in nature and not as restrictive.

### BRIEF DESCRIPTION OF THE DRAWINGS

Various aspects of apparatuses and methods will now be presented in the detailed description by way of example, and not by way of limitation, with reference to the accompanying drawings, wherein:

FIG. 1 is an illustration of a neuromodulation system including an implantable neuromodulation device and an external unit positioned relative to a body.

FIG. 2 is a block diagram of the neuromodulation system of FIG. 1.

FIG. 3 is a schematic illustration of components of the implantable neuromodulation device of FIG. 1.

FIG. 4 is another schematic illustration of components of the implantable neuromodulation device of FIG. 1.

FIG. 5 is an illustration of an example circuit architecture of components of the implantable neuromodulation device of FIG. 1.

FIG. 6 includes illustrations of stimulation pulses output by an implantable neuromodulation device based on a signal transmitted by the external unit.

FIG. 7 is an illustration of a signal sensed and recorded by the implantable neuromodulation system of FIG. 1 that includes a series of six stimulation responses, each elicited by the delivery of an electrical stimulation pulse at or near a nerve bundle.

FIG. 8A is an illustration of a stimulation pulse delivered by the implantable neuromodulation system of FIG. 1.

FIG. 8B is an illustration of stimulation artifact of a stimulation response elicited by the delivery of the electrical stimulation pulse of FIG. 8A.

FIG. 9 is an illustration of an evoked compound action potential elicited by the delivery of an electrical stimulation pulse of FIG. 8A.

FIG. 10 is a flowchart of a method quantifying the average energy in evoked compound action potentials in real-time.

FIG. 11A is a block diagram illustration of a deconvolution based inverse filter that may be used to implement the method of FIG. 10.

FIG. 11B is a block diagram illustration of an adaptive filter that may be used to implement the method of FIG. 10.

FIGS. 12A-12C are illustrations of three consecutive evoked stimulation responses (or evoked neural responses), each with a corresponding stimulation artifact and evoked compound action potential (not visible).

FIGS. 13A-13C are illustrations of the three consecutive evoked stimulation responses (or evoked neural responses) of FIG. 12 after filtering in accordance with the method of FIG. 10, wherein the presence of the stimulation artifacts is reduced, and the evoked compound action potentials are now visible.

FIG. 14 is an illustration of the mean squared error minima identifying the beginning of each evoked compound action potential for the three consecutive cycles of FIG. 13.

FIG. 15 is a flowchart of another method for quantifying the average energy in evoked compound action potentials in real-time.

FIG. 16 is an illustration of a running average of a set of evoked compound action potentials.

FIG. 17 is a block diagram illustration of a proportional-integral (PI) closed loop controller to maintain optimal stimulation amplitude.

FIG. 18 is a flowchart of a method of adjusting stimulation therapy delivered to tissue of a patient by an implanted neuromodulation device.

### DETAILED DESCRIPTION

Neurostimulation therapies are often limited due to a loss of therapeutic effect over time. Habituation and desensitization with increasing tolerance to the neurostimulation therapy is a considerable challenge in the treatment of disorders such as chronic pain and urinary dysfunction. This effect is exacerbated with the use of static stimulation waveforms. By applying multiple stimulation waveforms or a more stochastic dynamic pattern of waveform, the loss of therapeutic benefit can be delayed or potentially eliminated. However, to quantify the effect of stimulation some form of feedback is necessary to determine if sufficient neural recruitment of target neural fibers has been achieved. For example, neural recruitment may be evidenced by an evoked stimulation response (also referred to herein as a neural response) sensed at or near the target neural fibers and captured by a neuromodulation device. Failure to recruit the target neural fibers will provide no therapeutic benefits, whereas exceeding the upper limits of the therapeutic window can increase the risk of tissue damage over time and can cause pain. Therapeutic window (or treatment window), as used herein generally means the range between the perception by a patient of paresthesia sensation onset and discomfort from stimulation.

The current state of the art in peripheral nerve stimulation (PNS) to treat a range of chronic disorders, such as neuropathic pain and urinary disfunction, adopt open loop modalities, wherein the pulse generator output is a fixed waveform. While this approach is sufficient to provide therapeutic benefits, it is far from optimal, with adjustments to waveform amplitude performed by the patient. Variations in the electrical field at the nerve interface where the charge is delivered to a peripheral nerve fiber are common. Variations can be a result of changes in interface impedance, electrode movement and other interfering factors. Electrode movement can result from cardiac and respiratory motion or ambulation. This can impact the ability of the device to elicit an action potential (AP) response or provide the desired stimulation response consistently. Therefore, by dynamically adjusting stimulation doses based on feedback, consistent neural recruitment is possible. Stimulation dose, as used herein means the type and/or pattern of stimulation that is delivered in order to evoke a neural response at the target fibers. The type of stimulation may be, for example, electrical pulse waveform stimulation, where a pulse is defined by amplitude and pulse width. The pattern of stimulation may be a single pulse, a pulse train (a series of stimulation pulses separated by a brief time interval specified by a frequency parameter) or continuous stimulation for a period of time.

A means of stimulation feedback is highly desirable to optimize therapy delivery. Fundamentally, feedback to simply acknowledge neural recruitment could have significant benefits. One of the failures of occipital nerve stimulation (ONS) to treat migraine has been due to lead movement and migration resulting in a failure to recruit target neural fibers, while patients believed they were receiving therapy. Moreover, the introduction of new stimulation waveforms that stimulate at sub-paresthesia levels complicate open loop systems further, where it becomes increasingly more difficult for a patient to adjust their stimulation therapy effectively.

Evoked compound action potentials (ECAPs) (also referred to herein as evoked AP responses or just AP responses) generated through electrical stimulation are difficult to detect as they have a low amplitude relative to the stimulation artifact produced. Even with a recording system with a high signal-to-noise ratio (SNR), stimulation artifact is several orders of magnitude greater than the physiological response evidenced by ECAPs. Recording of ECAPs is less difficult when measuring downstream of stimulation sites. For example, if a target nerve fiber is stimulated 100 mm distance from the measuring site, and the type of nerve fiber has a slow propagation velocity, then the artifact generated by stimulation will have decayed by the time the evoked AP response reaches the measurement site. However, in PNS devices it is not possible to have a downstream measurement site. Therefore, in PNS devices measurement electrodes must reside adjacent to the stimulation electrodes, and as a result the ECAPs will therefore be obscured by stimulation artifact.

It is desirable to provide a neuromodulation system that senses evoked compound action potentials (ECAPs) relative to a target nerve, and uses ECAPs as feedback to determine if a stimulation therapy was successful in eliciting a neural response from the target nerve. It is also desirable to have in such a system, an implantable component that has sensing and stimulating electrodes adjacent each other as part of the same single array of contacts and a processing component, either implantable or external, configured to process neural responses sensed by the sensing electrodes to detect ECAPs.

Accordingly, in one aspect of the disclosure, a neuromodulation system records and measures physiologically elicited neural signals from electrodes adjacent to the stimulation electrodes. The neuromodulation system includes an implantable neuromodulation device with both sensing and stimulation electrodes as part of the same array. The electrodes can be in a cuff arrangement that is attached to the nerve or an array that resides alongside the nerve bundle. The implantable neuromodulation device and sensing electronics are hardwired and located at the nerve site.

The signals acquired from the sensing electrodes are processed in real-time to derive measures or biomarkers that may be used to control stimulation by the implantable neuromodulation device. To this end, the acquired signals may be transmitted to an externa unit that resides outside of the body. The acquired signals may correspond to electrical activity of neural tissue resulting from the delivery of a stimulation therapy by the implantable neuromodulation device. These electrical activity signals are referred to herein as stimulation response signals.

In some aspects the acquired stimulation response signals are processed by digital filters to remove stimulation artifact. Once stimulation artifact is removed the residual stimulation response signal is processed to locate single fiber action potentials (APs) or electrically evoked compound action potentials (ECAPs) wavelets. Measures or biomarkers may be derived from these action potentials and may be used as a means to quantify current stimulation therapy. In further aspects, stimulation therapy can be automatically controlled in real-time based on measures or biomarkers.

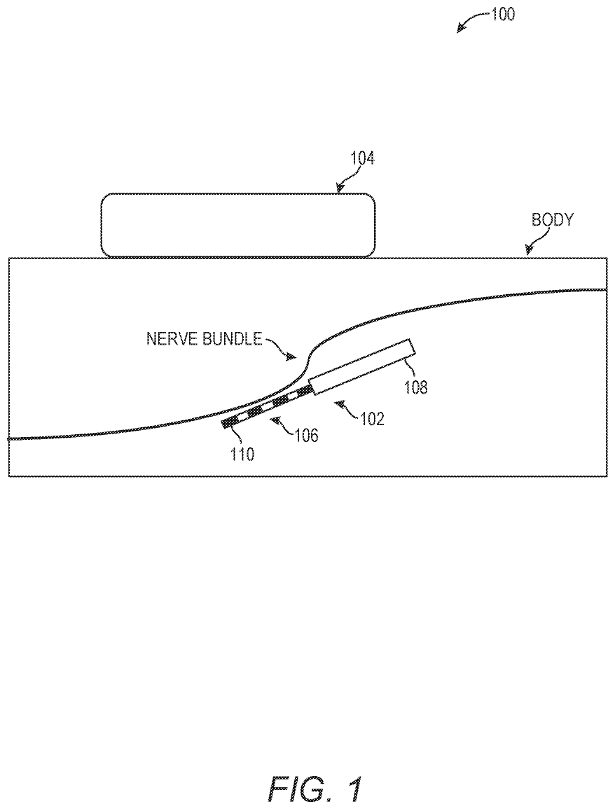

With reference to FIG. 1, disclosed herein is a wireless neuromodulation system 100. The neuromodulation system 100 includes an implantable neuromodulation device 102 configured to be implanted in a body adjacent a nerve bundle, and an external unit 104 configured to provide power to the implantable neuromodulation device, to receive and process signals from the implantable neuromodulation device, and to transmit stimulation information to the implantable neuromodulation device.

The implantable neuromodulation device 102 includes one or more electrode platforms 106 connected to an electronics component 108. The one or more electrode platforms 106 include a number of electrodes 110. While the example implantable neuromodulation device 102 shown in FIG. 1 includes a single electrode platform 106 with four electrode 110, an implantable neuromodulation device 102 may be configured with additional electrode platforms, and the electrode platforms may be configured with more or less than four electrodes.

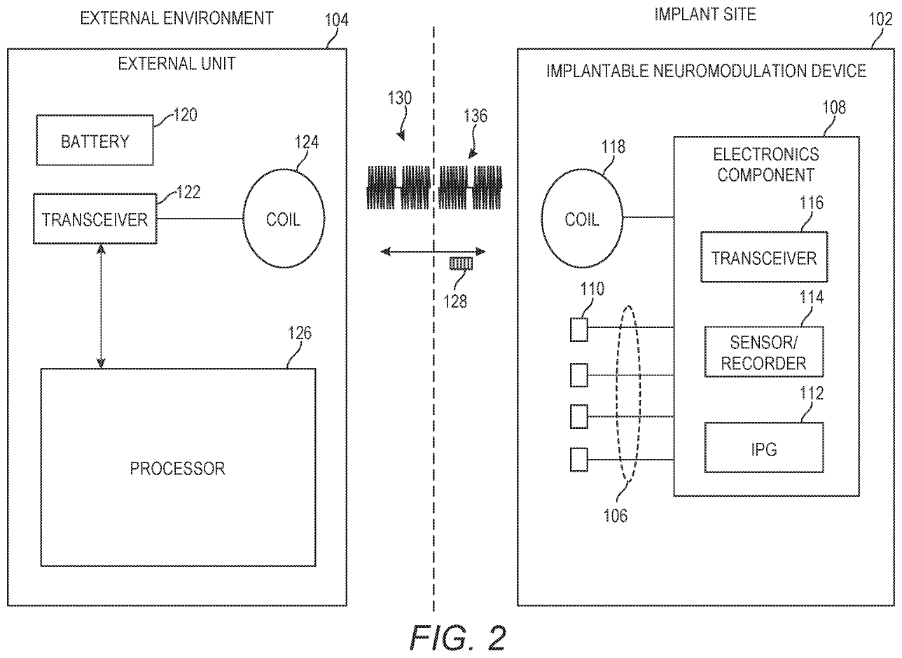

With reference to FIG. 2, the electronics component 108 of the implantable neuromodulation device 102 includes an application specific integrated circuit (ASIC) that includes circuitry that functions as an implantable pulse generator (IPG) 112 that generates and outputs stimulation therapy through the electrodes 110 of the one or more electrode platforms 106. The ASIC may include additional circuitry that functions as an electrophysiology signal sensor/recorder 114 that senses electrical activity of neural tissue captured through the electrodes 110 of the one or more electrode platforms 106 and stores records 128 of the electrical activity. The electronics component 108 also includes power and communication components, e.g., a transceiver 116 and an antenna 118. In an embodiment, the antenna 118 is a coil. The power and communication components are configured to transmit the records 128 of electrical activity to the external unit 104.

The external unit 104 includes a battery 120 and power and communications components, e.g., a transceiver 122 and an antenna 124. In an embodiment, the antenna 124 is a coil. The external unit 104 also includes a processor 126 (also referred to as a microcontroller). In some embodiments the processor 126 is configured to control the power and communications components, e.g., a transceiver 122 and an antenna 124, to transmit and modulate a carrier signal 130, e.g., a radio-frequency (RF) signal, in a way that provides power to the implantable neuromodulation device 102 and provides stimulation information that controls the delivery of neurostimulation by the implantable neuromodulation device 102.

In some embodiments, the stimulation information transmitted by the external unit 104 may specify neurostimulation settings or parameters, e.g., pulse width, pulse amplitude, etc., used by the implantable neuromodulation device 102 to deliver stimulation therapy. In this case, the stimulation information transmitted by the external unit 104 is used to program the implantable pulse generator 112 of the implantable neuromodulation device 102. In some embodiments, instead of being used to program the implantable pulse generator 112 of the implantable neuromodulation device 102, the stimulation information transmitted by the external unit 104 controls the delivery of neurostimulation therapy by the implantable neuromodulation device in real time. In this case, the stimulation information transmitted by the external unit 104 controls the timing of neurostimulation therapy and the neurostimulation settings or parameters, e.g., pulse width, pulse amplitude, etc., of the therapy.

The processor 126 of the external unit 104 is also configured to process neural information included in electrical activity of neural tissue sensed by the electrode platform 106 of the implantable neuromodulation device 102 and received by the external unit 104 from the implantable neuromodulation device. To this end, the processor 126 is configured to sense and analyze evoked compound action potentials (ECAPs) resulting from delivery of stimulation therapy by the implanted neuromodulation device, and to initiate changes in neuromodulation therapy to ensure delivery of effective therapy, e.g., therapy that results in neural recruitment, and to ensure effective sensing of electrical activity resulting from delivery of therapy.

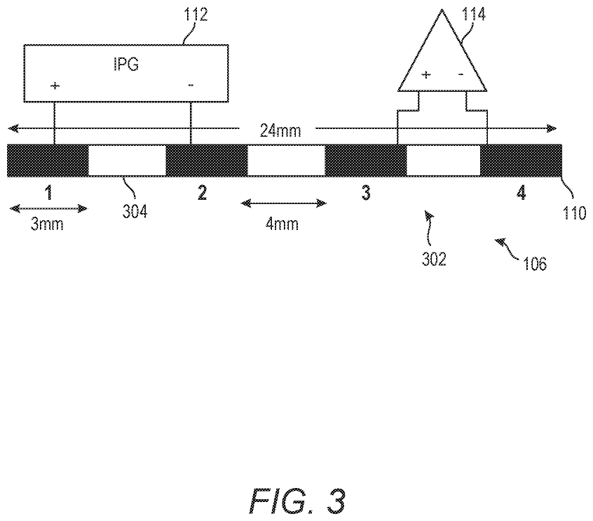

With reference to FIG. 3, in some embodiments the electrode platform 106 of the implantable neuromodulation device 102 includes an electrode-bearing body 302 having four electrodes 110. In some embodiments, the electrodes 110 are uniformly spaced apart and separated by non-conductive regions 304 of the electrode-bearing body 302.

In the configuration shown in FIG. 3, two electrodes 110 (electrode 1 and electrode 2) are coupled to the IPG 112 of the electronics component 108 for purposes of delivery electrical stimulation pulses to the body, and two electrodes 110 (electrode 3 and electrode 4) are coupled to the signal sensor/recorder 114 of the electronics component 108 for purposes of sensing and recording electrical activity of neural tissue resulting from the delivery of a stimulation therapy by the implantable neuromodulation device. Each possible combination of two electrodes 110 can be configured in unipolar and bipolar modes. In this example, electrodes 1 and 2 are configured for bipolar electrical stimulation and electrodes 3 and 4 are configured for bipolar recording. In other configurations utilizing one electrode as a reference with the remaining electrodes configured as working electrodes, unipolar signals can be recorded, thereby enabling multiple combinations of bipolar signals to be derived.

With reference to FIG. 2, in some embodiments the implantable neuromodulation device 102 is configured to receive radio-frequency (RF) signals 130 (also referred to as Tx signals or incident signals) from the external unit 104 and to output stimulation pulses to an electrode 110 based on the RF signals. In some embodiments the RF signals are in a medical device radiocommunication (MedRadio) frequency range, e.g., 401-406, 413-419, 426-432, 438-444, and 451-457 MHz. To this end, the IPG 112 of the implantable neuromodulation device 102 may be configured to produce stimulation pulses having different pulse energies based on the characteristics or configuration of the RF signal 130. For example, as disclosed later below the characteristics or configuration of the RF signal 130 may determine the widths and/or amplitudes (and thus the pulse energy) of a stimulation pulse. The implantable neuromodulation device 102 is also configured to harvest energy from the RF signals 130. Details on the generation and output of stimulation pulses and energy harvesting may be found in International Publication Number WO 2021/055146, entitled “Wirelessly Powered Stimulator.” Details may also be found in “A 430-MHz Wirelessly Powered Implantable Pulse Generator With Intensity/Rate Control and Sub-1 μA Quiescent Current Consumption,” Honeming Lyu et al, IEEE Transactions on Biomedical Circuits and Systems, Vol. 13, No. 1, February 2019.

The processor 126 of the external unit 104 is configured to control the operation of the transceiver 122 to generate an RF signal 130 having a configuration that determines stimulation pulses energy and to transmit the RF signal to the implantable neuromodulation device 102. As noted above, the configuration of the RF signal 130, in turn, controls a characteristic, e.g., width and/or amplitude, of the stimulation pulse produced by the IPG 112 of the implantable neuromodulation device 102. For example, with reference to FIG. 2, notches 136 can be included in the RF signal 130 to control the width of stimulation pulses, and to control the rate at which stimulation pulses are output by the IPG 112. A notch 136 may be included by reducing the amplitude of the RF signal 130 to a percentage of the peak amplitude that is used for purposes of energy harvesting by the implantable neuromodulation device 102.

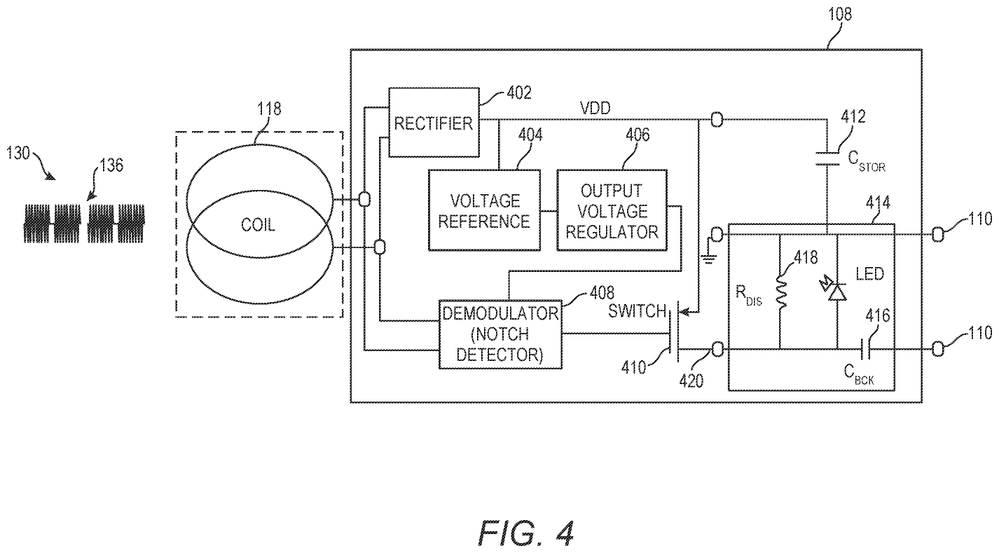

With reference to FIG. 4, in some embodiments, the electronics component 108 includes a rectifier 402, a voltage reference 404, an output voltage regulator 406, a demodulator 408, a switch 410, an energy storage capacitor 412, and stimulation circuitry 414. The electronics component 108 receives RF signals 130 through the antenna 118 of the implantable neuromodulation device 102. The rectifier 402 is configured to rectify the RF signal 130 to generate an output voltage (VDD). Depending on the open/closed state of the switch 410, the output voltage VDD is coupled to the energy storage capacitor 412 or to the stimulation circuitry 414. When coupled to the energy storage capacitor 412, the output voltage VDD charges the energy storage capacitor.

Continuing with reference to FIG. 4, the output voltage VDD provided by the rectifier 402 is compared to a voltage reference 404 through the output voltage regulator 406. To this end, the output voltage regulator 406 is configured to compare fractions of VDD with a constant voltage reference 404. In some configurations, when the output voltage VDD exceeds a first threshold value, e.g., 19/12 of the voltage reference 404, a discharge current path (not shown in FIG. 4) is enabled to discharge excess charge from the energy storage capacitor 412. When the output voltage VDD is less than a second threshold value, e.g., 19/16 of the voltage reference 404, the output voltage regulator 406 disables the demodulator 408. Circuitry within the output voltage regulator 406 sets the first and second threshold values of the voltage reference 404 to thereby regulate the amplitude of the stimulation pulse. For example, in one configuration, the amplitude may be regulated to be in the range of 2.7 volts and 3.6 volts.

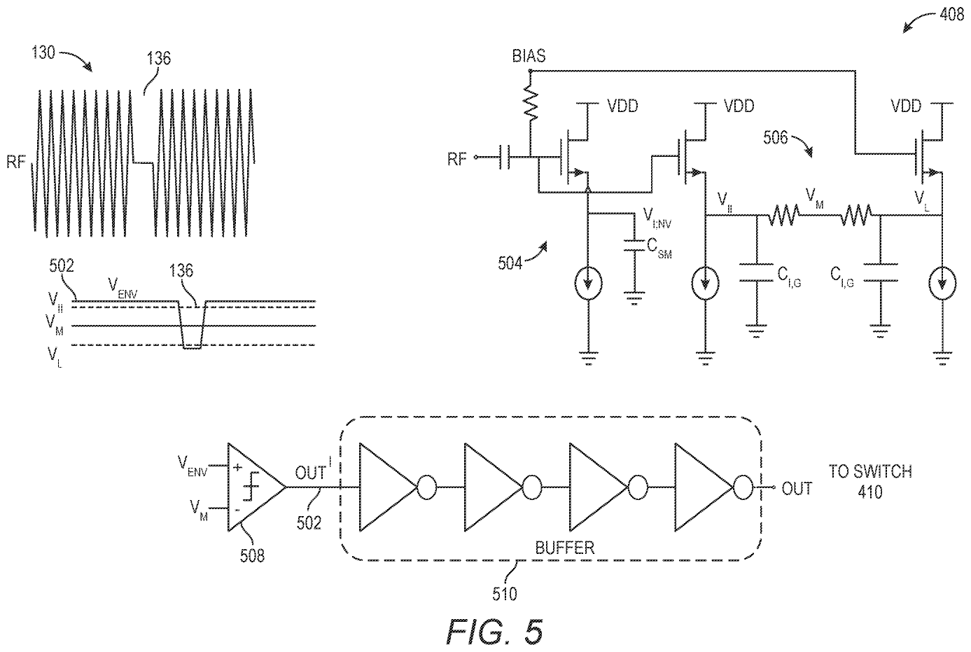

As shown in FIG. 4, the RF signals 130 are also received at the demodulator 408. The demodulator 408 is configured to process the RF signals 130 to control the on/off state of the switch 410. To this end, and with additional reference to FIG. 5, the demodulator 408 is configured to output a timing signal 502 that replicates the timing of notches 136 present in the Tx signal 130. In view of this functionality, the demodulator 408 may be referred to as a notch detector. The high end, low end, and transient envelope of the Tx signal 130 are denoted as V_{H}, V_{L}, and V_{ENV}, respectively in the timing signal 502. The Tx signal 130 is input to circuitry 504 of the demodulator that includes a V_{ENV} detection branch, a V_{H} detection branch, and a V_{L} detection branch. The V_{ENV} detection branch may use a relatively small capacitor C_{SM} to extract V_{ENV} from the Tx signal, while V_{H} and V_{L} can be extracted on larger capacitors with and without the AC input, respectively. The average V_{M}, e.g., the average of the high end V_{H} and the low end V_{L}, can be obtained through a resistive divider 506.

The average V_{M} is input to a comparator 508 and compared with V_{ENV} to reconstruct the timing of notches 136 included in the Tx signal 130. Capacitors C_{SM} and C_{LG} can be selected to be e.g., 100 fF and e.g., 36 pF, respectively. As C_{SM}<<C_{LG}, the average V_{M} can be considered as constant so that the discharging and charging of C_{SM} determines the delays from the starting point of a notch 136 and the ending point of a notch, respectively. The timing signal 502 at the output of the comparator 508 can then be sharpened by a following buffer 510 and then provided to the input of the switch 410.

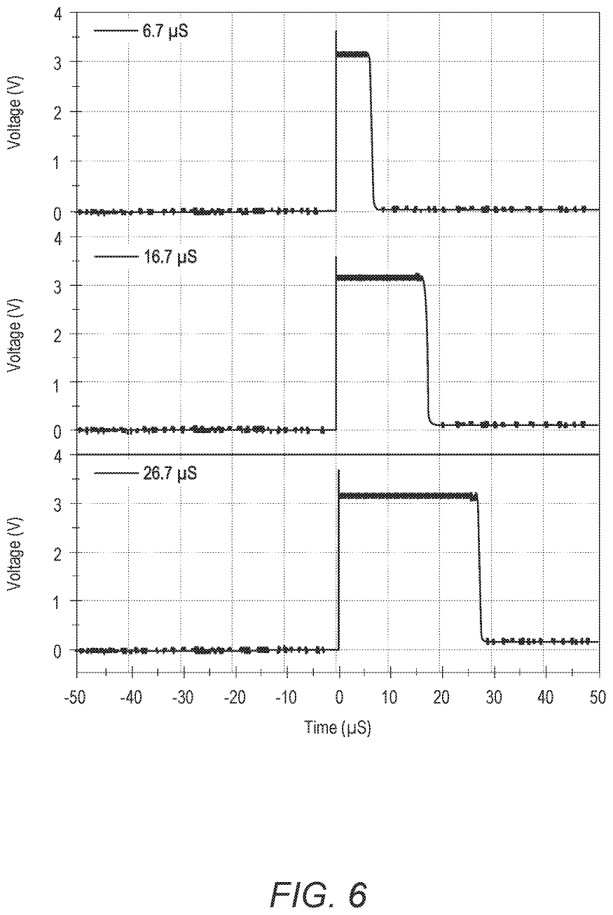

With continued reference to FIGS. 4 and 5, upon detection of a notch 136, the demodulator 408 sets the switch 410 to a closed state and holds the switch in the closed state until the notch is no longer detected. During the time the switch 410 is closed, the output voltage VDD and the energy storage capacitor 412 are coupled to the stimulation circuitry 414 and a stimulation pulse is generated and delivered through the electrodes 110. The duration of the notch 136 determines the pulse width of the stimulation pulse, and thus the pulse energy of the stimulation pulse. Examples of different stimulation pulses output by the IPG 112 of the implantable neuromodulation device 102 are shown in FIG. 6, which includes waveforms of 6.7 μs, 16.7 μs, and 26.7 μs stimulation pulses respectively triggered by 10 μs, 20 μs, and 30 μs notches.

Regarding the stimulation circuitry 414 of the electronics component 108, the circuitry includes a DC-block capacitor 416 and a discharge resistor 418. The DC-block capacitor 416 is coupled to the output 420 of the switch 410 and to the electrode 110. The DC-block capacitor 416 provides charge-neutralization and prevents any release of DC charge to the electrode 110. The discharge resistor 418 nulls the accumulated charge on C_{BCK}.

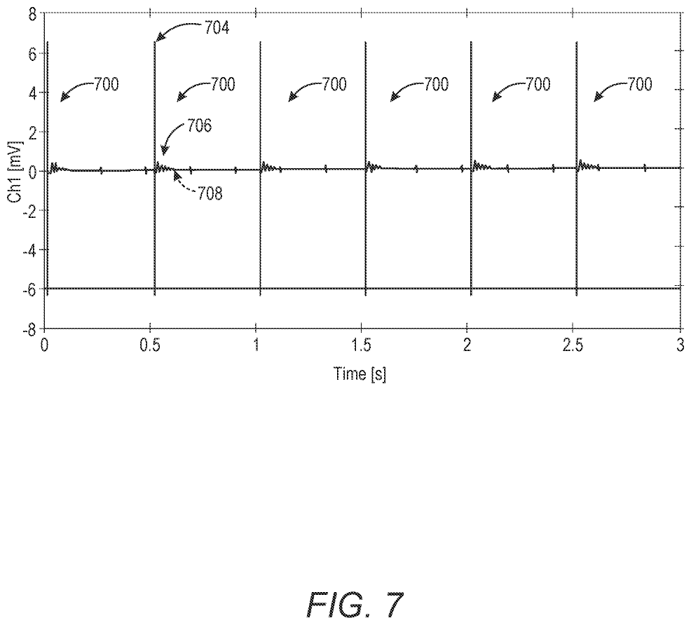

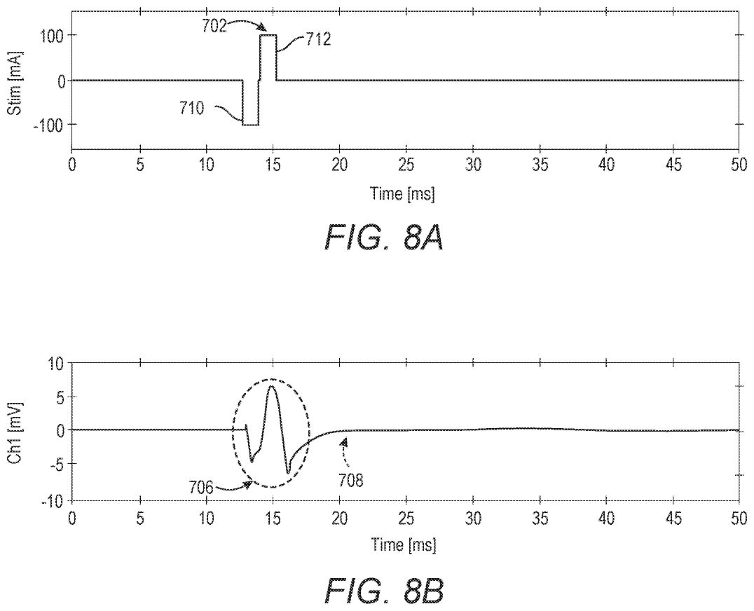

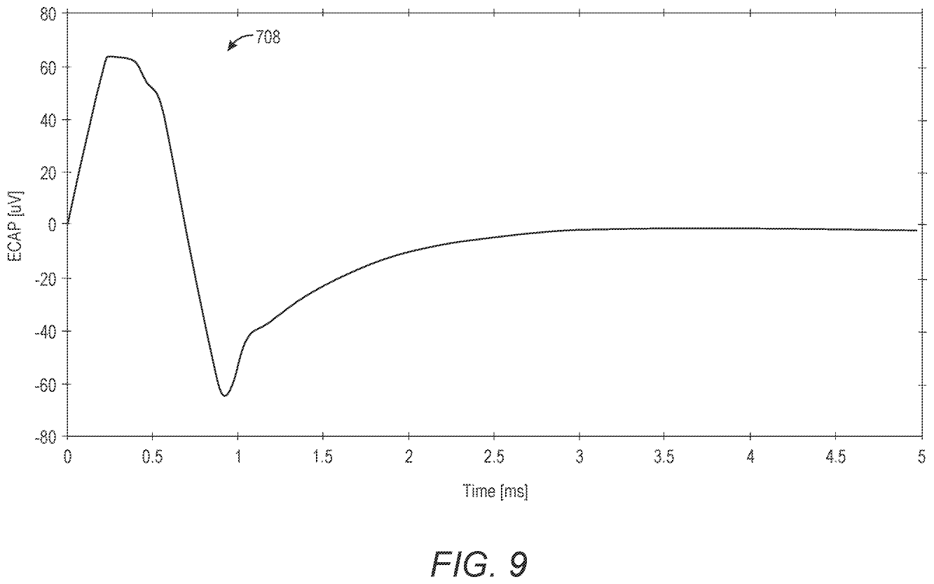

FIG. 7 illustrates a series of six stimulation responses 700, each elicited by the delivery of an electrical stimulation pulse at or near a nerve bundle. FIG. 8A illustrates one cathodic electrical stimulation pulse 702. FIG. 8B illustrates a zoomed in portion of a resultant stimulation artifact 706 of a stimulation response elicited by the delivery of the electrical stimulation pulse of FIG. 8A. FIG. 9 illustrates a typical AP response 708 elicited by the delivery of an electrical stimulation pulse of FIG. 8A.

With reference to FIG. 7, each of six stimulation responses 700 resulting from electrical activity sensed at the nerve bundle exhibit an evoked stimulation response 704, a resultant stimulation artifact 706, and an evoked compound action potential (ECAP) response 708 (not visible in FIG. 7 or 8, but shown in FIG. 9). ECAP responses 708, also referred to herein as action potential (AP) responses, occur uniformly, potentially under cardiac autonomic control. The significant difference in stimulation artifact 706 and AP response 708 amplitude is salient.

With reference to FIGS. 8A and 8B, stimulation artifact 706 is evident from both negative stimulus pulse 710 and positive charge recovery pulse 712. In the example of FIG. 8B an AP response 708 is generated at 20 ms, however, it is not identifiable in this example as the AP response is in the u V range and stimulation artifact 706 occupies the mV range. The AP response 708 occurs at 20 ms which is 7 ms from the onset of stimulation pulse 702. Therefore, the AP is propagating at ˜2 m/s which is common for Aδ or C fibers which require 7 ms to travel the 14 mm between electrodes as shown in FIG. 2.

With reference to FIG. 9, the typical AP response 708 elicited through stimulation is measured during a period with minimal artifact and noise. In this example the AP response 708 waveform is ˜60 μV peak-to-peak which is considerably lower amplitude than the stimulation artifact 706 shown in FIG. 8B.

Because the stimulation artifact 706 is proportional to the charge delivered, which is dependent on the pulse amplitude and width of the stimulation pulse 702, the stimulation artifact generated at increased amplitude can be predicted once the stimulation artifact characteristics recorded on the sensing electrodes are determined and modeled. This is crucial as any stimulation artifact 706 removal routine that is adapted in the presence of neural signals will likely attenuate or remove the neural components, e.g., the AP response 708, of interest.

In accordance with embodiments disclosed herein, a stimulation response signal, such as illustrated in FIG. 7, is processed to attenuate, or reduce the presence of stimulation artifacts 706 without attenuating the AP response 708. Once stimulation artifacts 706 are attenuated or filtered from the stimulation response signal, the remaining signal is processed to locate the AP response 708. As the stimulation (i.e., the delivery of electrical stimulation pulse 702) is periodic, once the AP response 708 to such stimulation is identified, the exact time lag between the evoked stimulation response 704 and the AP response 708 can be measured and used to predict the location of the AP response 708 after the delivery of each electrical stimulation pulse 702 (or after the detection of the evoked stimulation response 704).

Knowing the location or time of occurrence of the AP response 708 relative to the time of evoked stimulation response 704 is beneficial because signal processing can be applied to that particular time segment (the segment containing the AP response 708). The AP response 708 may be used to quantify the overall neural response to the electrical stimulation pulse 702. For example, if the electrical stimulation pulse 702 is too low there will not be a neural response. In other words, there would be no stimulation response 700 as shown in FIG. 7. As another example, if the electrical stimulation pulse 702 is too high, a high amplitude evoked stimulation response 704 within the stimulation response 700 may be noted, which may be outside of the treatment window. As previously mentioned, treatment window generally means the range between the perception by a patient of paresthesia sensation onset and discomfort from stimulation. In the case of a high amplitude evoked stimulation response 704 outside the treatment window, the patient would experience discomfort.

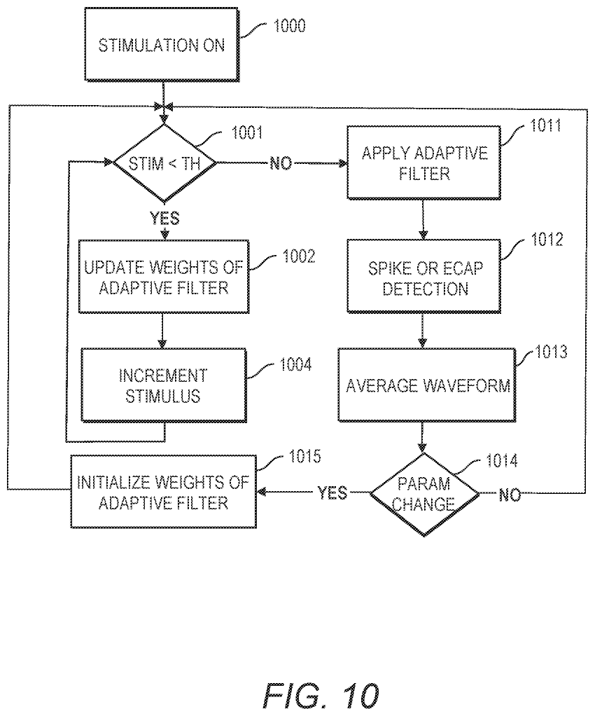

With reference to FIG. 10, a method of quantifying the average energy in evoked compound action potentials (ECAPs) in a signal representing sensed electrical activity of a nerve is now described. The method involves reducing the presence of stimulation artifact 706 in a signal representing sensed electrical activity of a nerve. The method may be performed by the implantable neuromodulation device 102 of FIG. 1, either alone or in conjunction with the external unit 104. The implantable neuromodulation device 102 is configured to acquire the signal representing sensed electrical activity of a nerve, and to deliver stimulation therapy.

At block 1000, an implanted neuromodulation device 102 is activated to output stimulation therapy in the form of electrical stimulation pulses 702. For example, when treating pain, a patient may switch the device on when pain is excessive. The stimulation pulses 702 delivered by the device are defined by stimulation parameters e.g., pulse width, pulse amplitude, etc.

At block 1001, the current parameters of the electrical stimulation pulses 702 are processed by the implanted neuromodulation device 102 to determine if a stimulation level provided by the stimulation parameters is below a known activation threshold that elicits an AP response 708. To this end, the device may calculate an energy level for the electrical stimulation pulses 702 based on the parameters of the pulse. The known activation threshold is predefined and typically determined for a patient during a clinical programming session. The activation threshold may be adjusted during follow-up clinical sessions.

If the stimulation level provided by the stimulation parameters of the electrical stimulation pulse 702 is not below the activation threshold (in other words, it is above the activation threshold and thus elicits an ECAP or AP response 708), the process enters a filtering operation or filtering mode. To this end, the process proceeds to block 1011, where a signal representing sensed electrical activity of a nerve and acquired by the implanted neuromodulation device 102 is processed by an adaptive filter. The acquired signal representing sensed electrical activity of a nerve may correspond to a stimulation response 700 shown in FIG. 7 or zoomed in portion of the signal like the one shown in FIG. 8B.

The main purpose of the filtering operation (block 1011) is to significantly attenuate the stimulation artifact 706 generated through stimulation. The ECAP signals or AP responses 708 can be better derived if they are separated from the stimulation artifact 706 that is typically orders of magnitude greater than the physiological signal of interest, as illustrated in FIG. 7. In one approach, a model is used to predict the location (time of appearance) of a stimulation artifact 706 in the recorded signal and perform a linear subtraction to thereby significantly attenuate or remove the stimulation artifact from the recorded signal.

As illustrated in FIG. 10, when an electrical stimulation pulse 702 is applied (block 1000) and is not below a threshold (block 1001) that would elicit a physiological response i.e., an AP response 708, the stimulation artifact 706 generated by a particular applied electrical stimulation pulse 702 having a pulse waveform is measured at the recording electrodes (electrodes 3 and 4 in FIG. 3). The medium (e.g., neural fiber) effects between the stimulating electrodes (electrodes 1 and 2 in FIG. 3) and recording electrodes (electrodes 3 and 4 in FIG. 3) is modeled. A typical filter that preforms this modeling in real-time is an adaptive filter where the pulse waveform of the electrical stimulation pulse 702 delivered by the stimulating electrodes (electrodes 1 and 2 in FIG. 3) is the input signal and the recorded signal sensed at the recording electrodes (electrodes 3 and 4 in FIG. 3) is the reference signal.

The adaptive filter is one where the filter weights are adjusted in real-time to minimize the error between the input signals (the pulse waveform of the electrical stimulation pulse 702) and reference signals as will be described below. If for example the adaptive filter has an FIR structure, then it can be described as follows where Nis the filter model order:

$$y [ k ] = {\sum }_{n = 0}^{N - 1} W [ n ] x [ k - n ]$$

At block 1012, the filtered signal output by the adaptive filter is processed to detect AP responses 708, where an AP response signal is detected for each stimulation response 700. The AP response 708 may correspond to a signal like the one shown in FIG. 9. Several methods can be employed to detect the AP response 708. Given the fast conduction of AP response 708 on most neural fibers, the response will appear during stimulation. The AP response 708 can be measured in a time window with a fixed duration from the onset of a stimulation pulse 702. Here the peak can simply be measured and used for titration on the next cycle. Methods such as cross-correlation techniques can be used to detect the AP response 708 waveform or spike, or simple threshold techniques. However, sufficient stimulation artifact 706 should be attenuated where the AP response 708 will be of greater amplitude than the stimulation artifact enabling a simple threshold detection approach.

At block 1013, the detected AP responses 708 are processed to derive an average AP response signal using known waveform averaging techniques. If sufficient stimulation artifact 706 is removed the AP response 708 will be clear in the average waveform calculated in block 1013. The average AP response signal may be used to derive a measure relative to the evoked stimulation response 704. For example, a measure of relative amplitude between the average AP response signal and an average evoked stimulation response 704 may be a basis for adjusting or changing one or more stimulation parameters, e.g., a different pulse-width, duty cycle or stimulation frequency of the stimulation therapy.

At block 1014, a determination is made to adjust one or more of the stimulation parameters defining the stimulation pulses 702 based on the measure obtained in the average waveform process of block 1013. In some embodiments, at least one of the plurality of stimulation parameters (e.g., pulse amplitude, pulse width, duty cycle, stimulation frequency) may be decreased in response to the measure being above a first threshold. The first threshold may be an amplitude indicative of a stimulation therapy that is outside the treatment window of the patient so as to cause discomfort or pain. This may occur when the measure is based on high amplitude ECAP responses. The first threshold may be determined and set based on patient feedback to different stimulation therapies during implant of the implantable neuromodulation device, subject to post-implant updating based on subsequent patient feedback.

In some embodiments, at least one of the plurality of stimulation parameters (e.g., pulse amplitude, pulse width, duty cycle, stimulation frequency) may be increased in response to the measure being below a second threshold. The second threshold may be an amplitude indicative of a stimulation therapy that does not sufficiently elicit neural activity. This may occur when the measure is based on low amplitude or no amplitude ECAP responses. The second threshold may be determined based on sensing and analysis of stimulation responses during implant of the implantable neuromodulation device, subject to post-implant updating based on subsequent sensing and analysis.

In some embodiments, the plurality of stimulation parameters may be left at current values in response to the measure being below the first threshold (so as not to cause patient discomfort or pain) and above the second threshold (so as to elicit sufficient neural activity).

If none of the stimulation parameters has changed the process returns to block 1001 where the stimulation parameters, e.g., pulse width, pulse amplitude, are reviewed again to confirm that the stimulation level provided by the stimulation parameters is below the activation threshold. The process then cycles through the filtering operation of blocks 1011, 1012, and 1013 where another signal representing sensed electrical activity of a nerve that is acquired by the device is processed.

Returning to block 1014, if one or more of the stimulation parameters has changed the process proceeds to block 1015 where the adaptive filter weights are reset or initialized. The filter weights are reset to account for changes to characteristics of the stimulation artifact 706 that result from changes to the stimulation parameters.

The process then returns to block 1001 where the adjusted set of stimulation parameters are reviewed to determine if the stimulation level provided by the stimulation parameters is below the activation threshold. For example, if stimulation parameters are changed, where the level of stimulus is decreased either by reducing the duty cycle or amplitude for instance, the new set of stimulation parameters may fail to provide enough energy to elicit a nerve response.

At any time during the process of FIG. 10, if the stimulation level is determined at block 1001 to be below the activation threshold, the process enters a learning operation or learning mode. To this end, the process proceeds to block 1002 where filter weights are updated. Algorithms such as a least mean squares (LMS) or recursive least square (RLS) algorithms are examples of routines to update weights of the adaptive filter (applied in block 1011) by minimizing the error between the predicted and measured signal. In a dynamic system where the medium, e.g., neural fibers, may change slightly, for example pressure on the tissue, the characteristics of the medium may change. Therefore, updating the filter weights in real-time when new data is available enhances the performance of the adaptive filter and provides better approximations of the stimulation artifact 706.

At block 1004, the stimulation level is increased by an incremental amount by adjusting one or more of the stimulation parameters. For example, the level of stimulus may be increased by increasing the duty cycle or amplitude for instance. The process returns to block 1001, where if the stimulation level is still below the activation threshold the process cycles through blocks 1002 and 1004 until the activation threshold is reached at block 1001. To ensure physiological information of interest is not filtered, the learning algorithm corresponding to the blocks 1002, 1004 loop is only applied when the stimulation levels are below the activation threshold for an AP response.

To adapt the filter characteristics when switching to filtering mode (blocks 1011, 1012 and 1013) when the stimulation level exceeds the activation threshold at block 1001, a stimulation model is updated which is used to calculate the filter gain for new stimulation settings. This stimulation model can be a simple scalar coefficient that is updated or can utilize a curve fitting function, such as a second order polynomial or linear regression method.

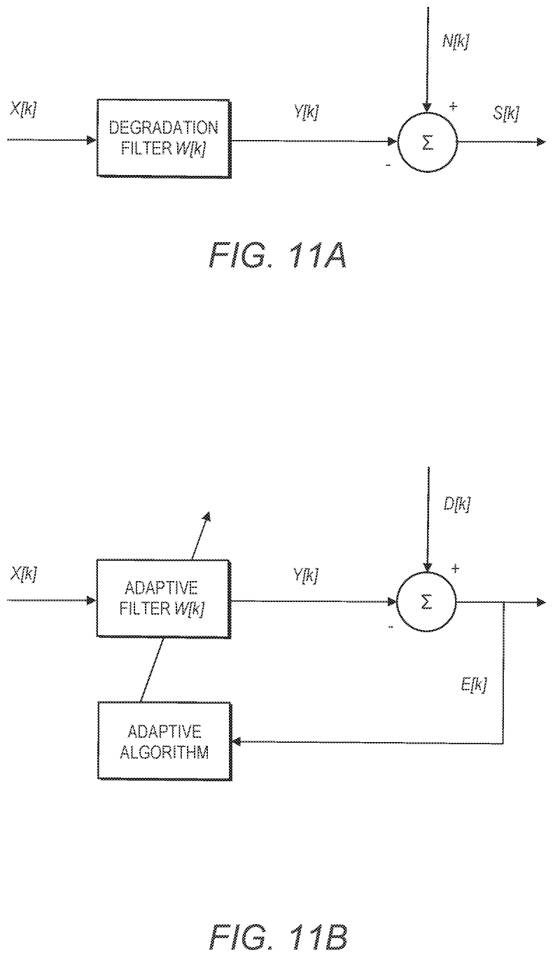

An example of an inverse filter to process a discrete time-series that can be used for fast initialization of a set of Finite Impulse Response (FIR) filter coefficients (used to initialize weights in block 1015) is illustrated in FIG. 11A. The input signal x[k] is convolved with the degradation filter w[k] and the residual is compared to the reference s[k] to calculate an error e[k]. A linear Weiner filter with weights w[k] can approximate the degradation effect from tissue as the stimulation pulse x[k] is applied to bipolar electrodes 1 and 2 discussed earlier. The causal FIR Wiener filter can adopt least squares estimate with input matrix X, output vector y, to determine filter weights derived as follows:

$$W = {( {X}^{T} X )}^{- 1} {X}^{T} y .$$

The output y[k] is the measured signal recorded from bipolar electrodes 3 and 4 described earlier. Such deconvolution filters are typically high-pass and can therefore amplify noise. In such circumstances it is advantageous to add noise to ensure a degree of noise reduction in the filter response by using s[k] to calculate the filter weights. The noise can be Gaussian or bandlimited Gaussian, where the noise signal n[k] is high pass filtered to create noise in the only the higher band, thereby creating attenuation at higher frequencies in the Wiener filter response, having a low pass effect. This requires a block estimation approach that is primarily suitable for initialization purposes.

An example of a real-time adaptive filter (applied in block 1011) is illustrated in FIG. 11B where the filter weights w[k] are adapted based on a learning algorithm such as the Least Means Square (LMS) or Recursive Least Square (RLS) methods. A cost function such as mean square error (MSE) is used to minimize the error between the filter residual y[k] and the desired signal d[k]. The desired signal is the measured stimulation artifact recorded between electrodes 3 and 4 in response to the stimulation waveform x[k]. When the MSE is at a minimum, the coefficients have converged, and the filter adequately models the characteristics of the tissue being stimulated. If the tissue changes due to electrode movement the filter will adapt. Convergence can occur quicker when initialized using the Wiener filter.

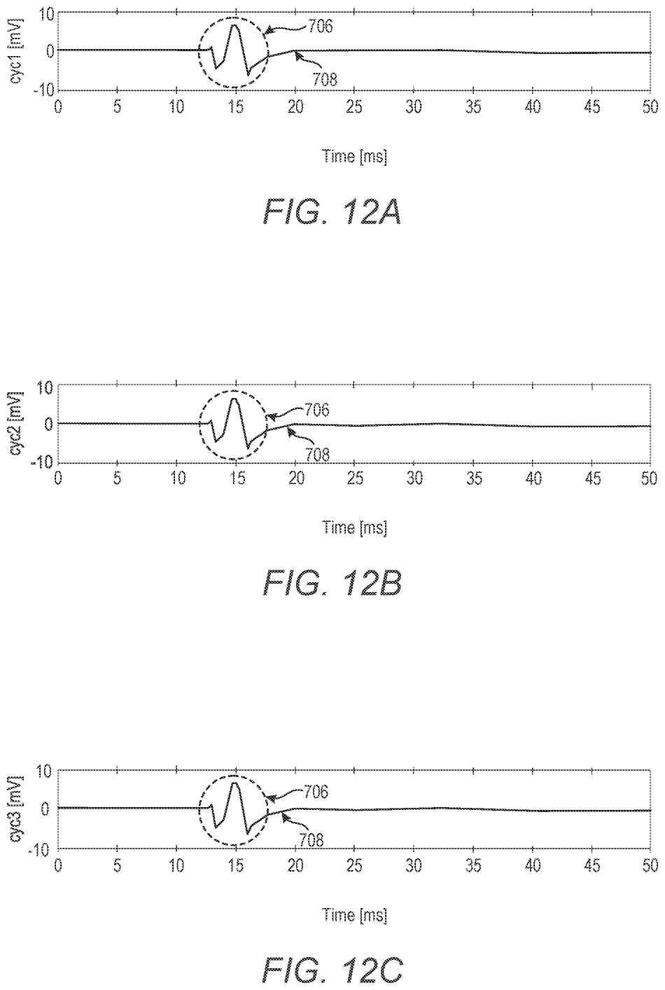

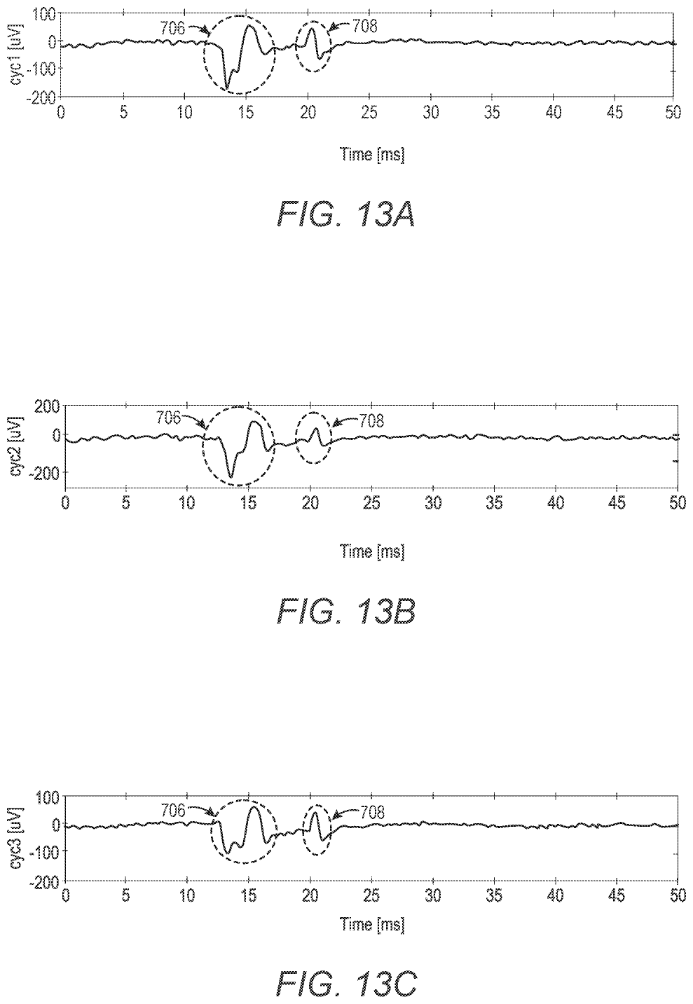

FIGS. 12A-12C are illustrations of three consecutive evoked stimulation responses (or evoked neural responses), each with a corresponding stimulation artifact and evoked compound action potential (not visible). FIGS. 13A-13C are illustrations of the three consecutive evoked stimulation responses (or evoked neural responses) of FIG. 12 after filtering in accordance with the method of FIG. 10, wherein the presence of the stimulation artifacts is reduced, and the evoked compound action potentials are now visible.

It is evident from FIGS. 12A-12C that no APs or ECAPs are visible, and the stimulation artifact 706 is approximately 7 mV peak-to-peak. With reference to FIGS. 13A-13C, the result of filtering the stimulation artifact 706 from the stimulation response 700 waveforms with the adaptive filter is present where the stimulation artifact has been reduced to approximately 100 μV peak-to-peak with a reduction of ˜70:1. The AP response 708 signals are now evident at 20 ms with similar amplitude, and at a level that is identifiable. The AP response 708 signals can now be processed through spike sorting or template matching algorithms that can be applied to the filter residual to determine the presence and location of the AP response 708 elicited through stimulation.

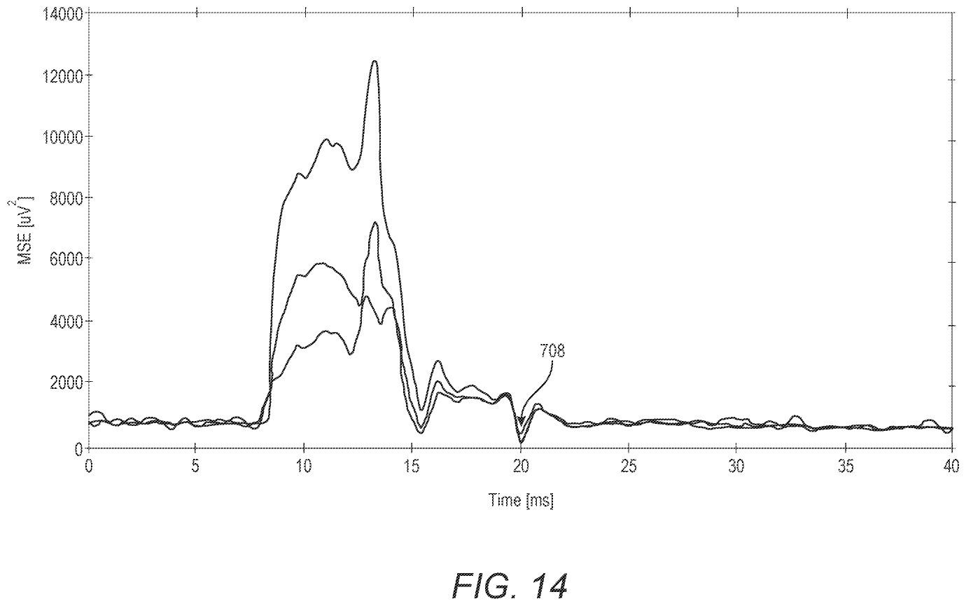

For example, the results of a template matching routine are illustrated in FIG. 14 for each of the 3 cycles presented, using a MSE measurement to determine fit and location. The MSE approach clearly identifies the AP response 708 location onset at 20 ms where the error is a minimum for each cycle. This approach could be enhanced by combining the MSE method with a cross-correlation function, although cross-correlation can be erroneously impacted by amplitude.

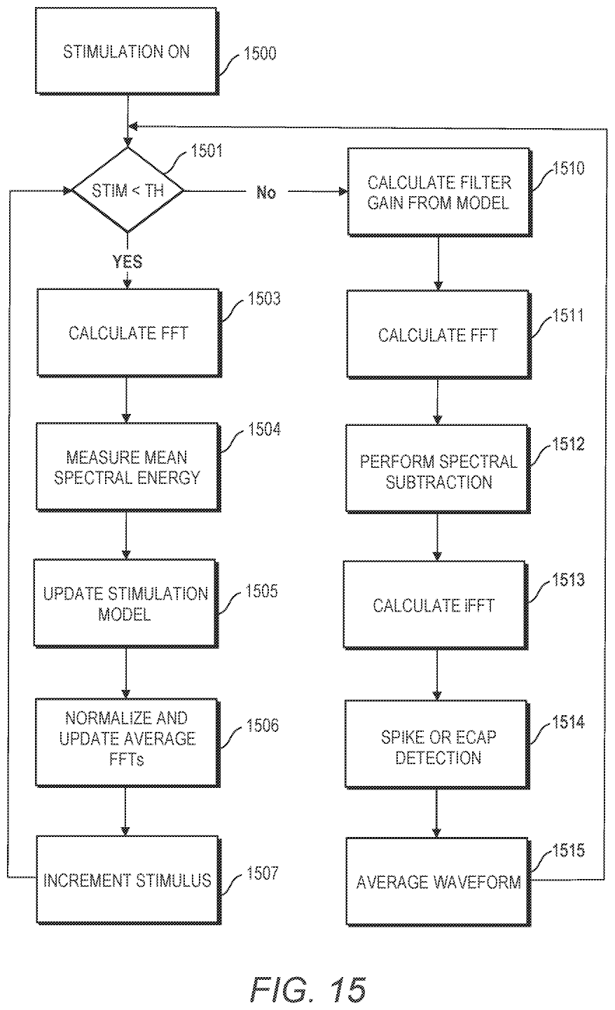

With reference to FIG. 15, another method of quantifying the average energy in AP response 708 in a signal representing sensed electrical activity of a nerve is now described. This method also involves reducing the presence of stimulation artifact 706 in a signal representing sensed electrical activity of a nerve.

In this method, a pseudo real-time approach is adopted that processes a segment of data as an alternative to a true real-time sample-by-sample strategy. This is acceptable as any adjustment to stimulation therapy can be performed in real-time prior to the next stimulation cycle. In circumstances where the frequency response of the medium (the neural fibers) being characterized is relatively static, it is often advantageous to perform filtering in the frequency domain, where instantaneous errors are eliminated.

At block 1500, an implanted neuromodulation device 102 is activated to output stimulation therapy in the form of electrical stimulation pulses 702. For example, when treating pain, a patient may switch the device on when pain is excessive. The stimulation pulses 702 delivered by the device are defined by stimulation parameters e.g., pulse width, pulse amplitude, etc.

At block 1501, the current parameters of the electrical stimulation pulses 702 are processed by the device to determine if a stimulation level provided by the stimulation parameters is below a known activation threshold that elicits an AP response 708. To this end, the device may calculate an energy level for the electrical stimulation pulses 702 based on the parameters of the pulse. The known activation threshold is predefined and typically determined for a patient during a clinical programming session. The activation threshold may be adjusted during follow-up clinical sessions.

If the stimulation level provided by the stimulation parameters of the electrical stimulation pulse 702 is below the activation threshold (in other words, it is not above the activation threshold and thus does not elicit an AP response), the process proceeds to block 1503 where a Fast Fourier Transform (FFT) is applied to an acquired stimulation response 700 signal to provide a frequency spectrum representation of the stimulation response signal.

At block 1504, the mean energy of the frequency spectrum is calculated and stored.

At block 1505, a stimulation model is adapted. The stimulation model is used to estimate the peak spectral energy based on the amplitude of the stimulus when attempting to filter the artifact. A spectral subtraction must be performed using an artifact spectrum that is obtained below the activation threshold to ensure the physiologic information is not present and therefore will not be removed following subtraction.

At block 1506, the frequency spectra are averaged and normalized. This process has the effect of increasing the artifact to random noise ratio.

At block 1507, one or more parameters of the stimulation therapy are changed in order to increase the stimulus being delivered, and the process returns to block 1501. The loop of blocks 1503, 1504, 1505, 1506, 1507, 1501 is repeated until the stimulation level provided by the stimulation parameters of the electrical stimulation pulse 702 is not below the activation threshold (in other words, it is above or exceeds the activation threshold and thus elicits an AP response), at which point the process proceeds to block 1510.

At block 1510, the stimulation model of block 1504 provides the spectral filter gain to block 1510. As the mean spectral energy has been calculated for each increment in stimulus (at block 1507) under the activation threshold, a simple model can be used (at block 1505) to estimate the peak amplitude of artifact in the frequency domain with the current stimulus above the activation threshold. This could be a simple extrapolation.

At block 1511 an acquired stimulation response 700 signal that includes physiologic information and stimulation artifact 706 is transformed into the frequency domain by FFT.

At block 1512, the normalized artifact averaged spectrum calculated in block 1505 is subtracted from the physiologic spectrum of block 1511.

At block 1513, the resultant is transformed back to the time domain by inverse FFT.

At block 1514, the time domain signal resulting from inverse FFT (block 1513) is processed to detect AP responses 708, where an AP response signal is detected for each stimulation response 700. The AP response 708 is detected based on a known time offset from the evoked stimulation response 704. The AP response 708 may correspond to a signal like the one shown in FIG. 9.

At block 1515, the AP responses 708 detected in the block 1514 are processed to derive an average AP response signal using known waveform averaging techniques. The average AP response signal may be used to derive a measure relative to the evoked stimulation response 704. For example, a measure of relative amplitude between the average AP response signal and an average evoked stimulation response 704 may be a basis for adjusting or changing one or more stimulation parameters, e.g., a different pulse-width, duty cycle or stimulation frequency of the stimulation therapy.

In the method of FIG. 15, the loop taken when no AP response is elicited (outcome at block 1501 is “yes”) estimates the stimulation-based degradation frequency spectrum (output of block 1503). This estimated frequency spectrum is subsequently used in the loop taken when an AP response is elicited (outcome at block 1501 is “no”) when at block 1512 the estimated spectrum from block 1503 is subtracted from the spectrum of the acquired stimulation response (output of block 1511) to estimate the physiologic spectrum. The degradation spectrum (output of block 1503) is multiplied by a gain function (block 1510) based on the stimulation model (block 1505) prior to spectral subtraction. At block 1513, the resultant spectrum is transformed back to the time-domain block by combining the filtered spectrum with the original phase of the recorded waveform, i.e., the acquired stimulation response signal.

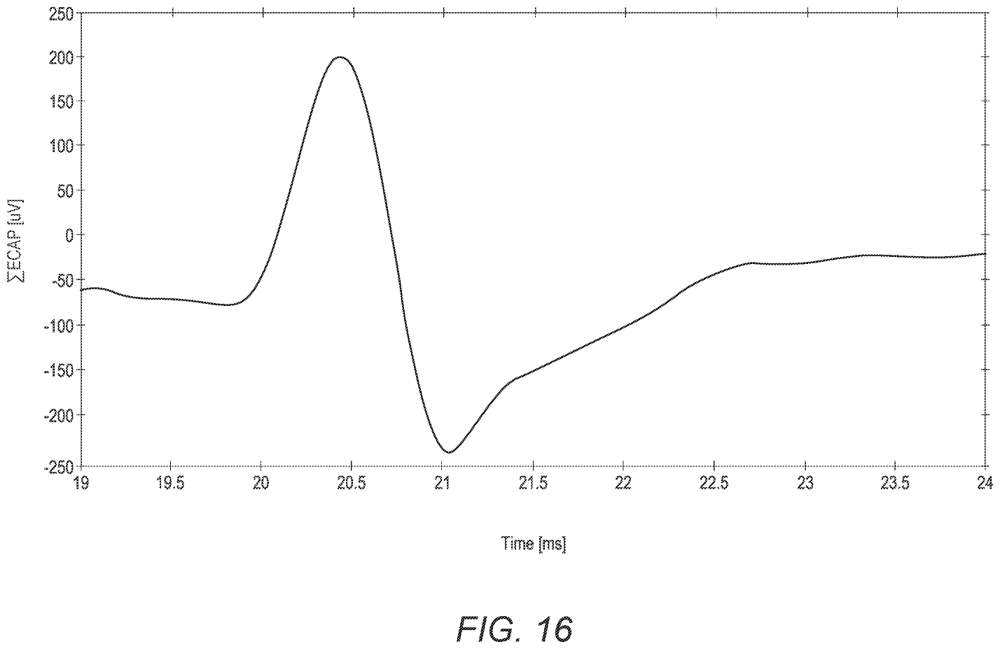

Regarding blocks 1013 and 1515, an example of averaging detected AP response 708 signals is illustrated in FIG. 16. This represents five averaged cycles of the detected AP responses 708 at 20 ms. Applying a running average function enhances the SNR for every cycle and attenuates random noise and artifact. This averaged waveform can be used as a biomarker to evaluate the physiological response to stimulation.

The average waveform biomarker described above can provide biofeedback on response from stimulation. Such a marker can alert a patient to zones that could cause pain from stimulus, or conversely not provide a therapeutic effect, or fail to elicit a response at all. Keeping stimulation pulse 702 delivery within the therapeutic window is critical. Here stimulus can be automated by utilizing this biomarker as a process variable to maintain homeostasis by keeping titration levels at the optimal dose response level. An adaptive controller can be used that adjusts the level of stimulation based on this feedback to keep the response at the ideal desired level or setpoint. The stimulation is adapted based on the average waveform amplitude of AP responses 708.

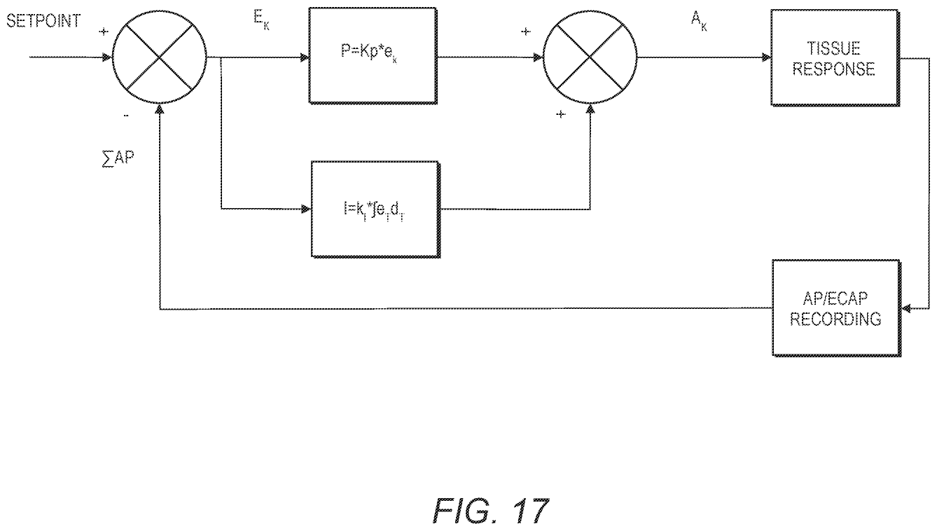

A classic Proportional-Integral (PI) controller, as illustrated in FIG. 17, is an example of an approach to maintain the desired AP response 708 amplitude elicited from a stimulation pulse 702 at a controller setpoint. The error which is the difference between the average AP biomarker ZAP and the setpoint is multiplied by a proportional gain Kp, which adjusts the instantaneous error in proportion to the desired response. A running average of the error is also multiplied by the integral gain KI and summed with the P contribution. The adjusted stimulation amplitude Ak for the kth cycle is applied and resultant AP response 708 signal recorded and the biomarker ZAP calculated.

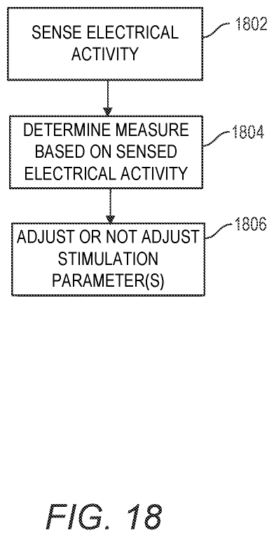

FIG. 18 is a flowchart of a method of adjusting stimulation therapy delivered to tissue of a patient by an implanted neuromodulation device. In some embodiments, the method is performed by a neuromodulation system 100 that includes the implanted neuromodulation device 102 and an external unit 104, such as shown in FIGS. 1 and 2. In some embodiments, the method is performed entirely by the implanted neuromodulation device 102.

At block 1802, electrical activity of the tissue resulting from a delivery of a stimulation therapy to the tissue through the implanted neuromodulation device 102 is sensed. For example, the electrical activity may be sensed by the implanted neuromodulation device 102. This step of the method is referred to herein as “step a.” The stimulation therapy delivered through the implanted neuromodulation device 102 is defined by a plurality of stimulation parameters, and the sensed electrical activity includes a stimulation response 700 having an evoked stimulation response 704, a stimulation artifact 706, and ideally an ECAP response 708.

At block 1804, a measure based on the ECAP response 708 and the evoked stimulation response 704 is determined. This step of the method is referred to herein as “step b.” With reference to the series of blocks 1011/1012/1013 of FIG. 10, in some embodiments, a measure based on the ECAP response 708 and the evoked stimulation response 704 is determined by obtaining an amplitude of the ECAP response 708, obtaining an amplitude of the evoked stimulation response 704, and calculating a relationship between the amplitude of the ECAP response and the amplitude of the evoked stimulation response. For example, the relationship may be a ratio of the amplitudes, such as an ECAP-amplitude/stimulation-response-amplitude ratio.

An amplitude of the ECAP response 708 may be obtained by applying a filter to the stimulation response (block 1011) and locating the ECAP response 708 in the filtered stimulation response (block 1012). The ECAP response 708 may be located by locating the ECAP response 708 relative to the evoked stimulation response 704 based on a known time offset. As described above with reference to FIG. 10, the known time offset may be determined based on previously sensed electrical activity of the tissue resulting from stimulation therapy.

Once the ECAP response 708 is located in the filtered stimulation response (block 1012), the amplitude of the ECAP response 708 can be derived from a waveform corresponding to the ECAP response. The amplitude of the ECAP response 708 may be derived from the waveform corresponding to the ECAP response 708 by calculating an average waveform from a plurality of ECAP responses (block 1013), and deriving the amplitude from the average waveform.

Regarding the filter that is applied to the stimulation response (block 1011), in some embodiments this filter is an adaptive filter having a plurality of filter weights that may be updated as needed. For example, in some instances an ECAP response 708 may not be present in the stimulation response 700. This may occur, for example, when the stimulation therapy is below an activation threshold that elicits an ECAP response 708 for the patient (block 1001 outcome is “Yes”). In this case, the filter weights are updated. In some embodiments, the filter-weight update process is the loop of blocks 1002/1004/1001 in FIG. 10, wherein the filter weights are updated (block 1002) and one or more of the plurality of stimulation parameters is incrementally increased (block 1004) until the stimulation therapy is not below the activation threshold that elicits an ECAP response 708 for the patient (block 1001 outcome is “No”).

At block 1806, after a measure based on ECAP response 708 is determined, a determination is made to adjust or not adjust one or more of the plurality of stimulation parameters of the stimulation therapy based on the measure (block 1014). The step of the method is referred to herein as “step c.” In some embodiments, at least one of the plurality of stimulation parameters (e.g., pulse amplitude, pulse width, etc.) is decreased in response to the measure being above a first threshold. As previously described, the first threshold may be an amplitude indicative of a stimulation therapy that is outside the treatment window of the patient so as to cause discomfort or pain. In some embodiments, at least one of the plurality of stimulation parameters (e.g., pulse amplitude, pulse width, etc.) is increased in response to the measure being below a second threshold. As previously described, the second threshold may be an amplitude indicative of a stimulation therapy that does not elicit neural activity (e.g., an ECAP response 708 for the patient). In some embodiments, the stimulation parameters are left at current values in response to the measure being below the first threshold and above the second threshold.

As previously described with reference to block 1804, in some embodiments the filter that is applied to the stimulation response (block 1011) to obtain the ECAP response 708 is an adaptive filter having a plurality of filter weights. In some embodiments, these filter weights are reset (block 1015) depending on the outcome of block 1806. For example, the filter weights may be reset when at least one of the plurality of stimulation parameters (e.g., pulse amplitude, pulse width, etc.) is decreased. The filter weights may also be reset when at least one of the plurality of stimulation parameters (e.g., pulse amplitude, pulse width, etc.) is increased.

As previously described, in some embodiments, the method of FIG. 18 is performed by a neuromodulation system 100 that includes an implanted neuromodulation device 102 and an external unit 104. In one such an embodiment, the implanted neuromodulation device performs step a (block 1802), while an external unit performs step b (block 1804) and step c (block 1806). As part of step a (block 1802), the implanted neuromodulation device 102 transmits the sensed signal corresponding to the sensed electrical activity and the external unit 104 receives the sensed signal. The implanted neuromodulation device 102 may also deliver the stimulation therapy to tissue. Also as previously described, in some embodiments, the method of FIG. 18 is performed entirely by an implantable neuromodulation device 102.

The various aspects of this disclosure are provided to enable one of ordinary skill in the art to practice the present invention. Various modifications to exemplary embodiments presented throughout this disclosure will be readily apparent to those skilled in the art. Thus, the claims are not intended to be limited to the various aspects of this disclosure but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

1. A neuromodulation system comprising:
an implantable neuromodulation device comprising:
an electrode platform;
an pulse generator coupled to the electrode platform and configured to output a stimulation therapy through the electrode platform, wherein the stimulation therapy is defined by a plurality of stimulation parameters,
a sensor/recorder coupled to the electrode platform and configured to:
process the plurality of stimulation parameters to determine if the stimulation therapy is above a known activation threshold that elicits an evoked compound action potential (ECAP) response,
in response to the stimulation therapy being above the known activation threshold, sense electrical activity of tissue resulting from a delivery of a stimulation therapy to the tissue, wherein the sensed electrical activity includes a stimulation response comprising an evoked stimulation response, a stimulation artifact and an ECAP response,
in response to the stimulation therapy being below the known activation threshold, refrain from sensing electrical activity of the tissue resulting from the stimulation therapy, and adjusting one or more of the plurality of stimulation parameters until the stimulation therapy is above a known activation threshold; and
a transceiver coupled to the sensor/recorder and configured to transmit a sensed signal corresponding to the electrical activity; and
an external unit comprising:
a transceiver configured to receive the sensed signal from the implantable neuromodulation device; and
a processor configured to determine a measure based on the ECAP response and the evoked stimulation response; and to determine whether to adjust one or more of the plurality of stimulation parameters of the stimulation therapy based on the measure.

2. The neuromodulation system of claim 1, wherein to determine a measure based on the ECAP response and the evoked stimulation response, the processor is configured to:
obtain an amplitude of the ECAP response; and
obtain an amplitude of the evoked stimulation response,
wherein the measure is a relationship between the amplitude of the ECAP response and the amplitude of the evoked stimulation response.

3. The neuromodulation system of claim 2, wherein to obtain an amplitude of the ECAP response, the processor is configured to:
apply a filter to the stimulation response;
locate the ECAP response in the filtered stimulation response; and
derive the amplitude from a waveform corresponding to the ECAP response.

4. The neuromodulation system of claim 3, wherein to locate the ECAP response, the processor is configured to locate the ECAP response relative to the evoked stimulation response based on a known time offset.

5. The neuromodulation system of claim 4, wherein the processor is further configured to determine the known time offset based on previously sensed electrical activity of the tissue resulting from the stimulation therapy.

6. The neuromodulation system of claim 3, wherein to derive the amplitude from a waveform corresponding to the ECAP response, the processor is configured to:
calculate an average waveform from a plurality of ECAP responses; and
derive the amplitude from the average waveform.

7. The neuromodulation system of claim 3, wherein the filter is an adaptive filter having a plurality of filter weights, and the processor is further configured to, in response to the stimulation therapy being below an activation threshold that elicits an ECAP response:
update the plurality of filter weights.

8. The neuromodulation system of claim 7, wherein to update the plurality of filter weights the processor is configured to:
incrementally increase at least one of the plurality of stimulation parameters and update the plurality of filter weights until the stimulation therapy is not below the activation threshold that elicits an ECAP response.

9. The neuromodulation system of claim 2, wherein to determine whether to adjust one or more of the plurality of stimulation parameters of the stimulation therapy based on the measure, the processor is configured to:
decrease at least one of the plurality of stimulation parameters in response to the measure being above a first threshold;
increase at least one of the plurality of stimulation parameters in response to the measure being below a second threshold; and
leave the plurality of stimulation parameters at current values in response to the measure being below a first threshold and above the second threshold.

10. The neuromodulation system of claim 9, wherein the processor is further configured to, in response to one of decreasing at least one of the stimulation parameters or increasing at least one of the stimulation parameters, resetting one or more of a plurality of filter weights of an adaptive filter configured to be applied to the stimulation response.

11. (canceled)

12. A method of adjusting stimulation therapy delivered to tissue of a patient by an implanted neuromodulation device, wherein the stimulation therapy is defined by a plurality of stimulation parameters, the method comprising:
a) processing the plurality of stimulation parameters to determine if the stimulation therapy is above a known activation threshold that elicits an evoked compound action potential (ECAP) response;
b) in response to the stimulation therapy being above the known activation threshold:
i) sensing electrical activity of the tissue resulting from a delivery of a stimulation therapy to the tissue through the implanted neurostimulation device, wherein the sensed electrical activity includes a stimulation response comprising an evoked stimulation response, a stimulation artifact and an ECAP response;
ii) determining a measure based on the ECAP response and the evoked stimulation response; and
iii) determining whether to adjust one or more of the plurality of stimulation parameters of the stimulation therapy based on the measure; and
c) in response to the stimulation therapy being below the known activation threshold:
i) refraining from sensing electrical activity of the tissue resulting from the stimulation therapy; and
ii) adjusting one or more of the plurality of stimulation parameters until the stimulation therapy is above a known activation threshold.

13. The method of claim 12, wherein determining a measure based on the ECAP response and the evoked stimulation response comprises:
obtaining an amplitude of the ECAP response; and
obtaining an amplitude of the evoked stimulation response,
wherein the measure is a relationship between the amplitude of the ECAP response and the amplitude of the evoked stimulation response.

14. The method of claim 13, wherein obtaining an amplitude of the ECAP response comprises:
applying a filter to the stimulation response;
locating the ECAP response in the filtered stimulation response; and
deriving the amplitude from a waveform corresponding to the ECAP response.

15. The method of claim 14, wherein locating the ECAP response comprises locating the ECAP response relative to the evoked stimulation response based on a known time offset.

16. The method of claim 15, further comprising determining the known time offset based on previously sensed electrical activity of the tissue resulting from the stimulation therapy.

17. The method of claim 14, wherein deriving the amplitude from a waveform corresponding to the ECAP response comprises:
calculating an average waveform from a plurality of ECAP responses; and
deriving the amplitude from the average waveform.

18. The method of claim 14, wherein the filter is an adaptive filter having a plurality of filter weights, and further comprising, in response to the stimulation therapy being below an activation threshold that elicits an ECAP response for the patient:
updating the plurality of filter weights.

19. The method of claim 18, wherein updating the plurality of filter weights comprises:
incrementally increasing at least one of the plurality of stimulation parameters and updating the plurality of filter weights until the stimulation therapy is not below the activation threshold that elicits an ECAP response for the patient.

20. The method of claim 13, wherein determining whether to adjust one or more of the plurality of stimulation parameters of the stimulation therapy based on the measure comprises:
decreasing at least one of the plurality of stimulation parameters in response to the measure being above a first threshold;
increasing at least one of the plurality of stimulation parameters in response to the measure being below a second threshold; and
leaving the plurality of stimulation parameters at current values in response to the measure being below a first threshold and above the second threshold.

21. The method of claim 20, further comprising, in response to one of decreasing at least one of the stimulation parameters or increasing at least one of the stimulation parameters, resetting one or more of a plurality of filter weights of an adaptive filter configured to be applied to the stimulation response.

22. The method of claim 12, wherein:
steps a, bi, ci, and cii are performed by the implanted neuromodulation device; and
steps bii and biii are performed by an external unit; and
further comprising transmitting, by the implanted neuromodulation device, a sensed signal corresponding to the sensed electrical activity.

23. (canceled)

24. (canceled)

25. (canceled)

26. (canceled)

27. (canceled)

28. (canceled)

29. (canceled)

30. (canceled)

31. (canceled)
